(19) United States
(12) Patent Application Publication
(10) Pub. No.: US 2006/0235330 A1
(43) Pub. Date: Oct. 19, 2006

(54) APPARATUS AND METHOD OF IDENTIFYING AND MANAGING MOOD

(76) Inventor: Michael E. HUFF, Pasadena, CA (US)

Correspondence Address: STARKWEATHER & ASSOCIATES, 9035 SOUTH 1300 EAST, SUITE 200, SANDY, UT, 84094, US

(21) Appl. No.: 11/163,180

(22) Filed: Oct. 7, 2005

Related U.S. Application Data
(60) Provisional application No. 60/594,499, filed on Apr. 13, 2005.

(51) Int. Cl. A61B 13/00

(52) U.S. Cl. 600/558; 600/559

(57) ABSTRACT

An apparatus for identifying and managing mood, including from seven to ten selectable mood buttons, each representing a mood; selectable trigger buttons, each representing a trigger for the mood; mood suggestions displayed after selection of a mood button and a trigger button; a speaker; and a mode selection button. The apparatus may include a wristband. The apparatus may include an on/off switch. The apparatus of may include an AC adapter jack. The mood suggestions may be displayed audibly through the speaker. Also disclosed is a method of identifying and managing mood, comprising the steps of displaying a selectable mood button, representing a mood; displaying a selectable trigger button, representing a trigger for the mood; and displaying mood suggestions.

### BACKGROUND OF THE INVENTION

This invention claims priority, under 35 U.S.C. §120, to the U.S. Provisional Patent Application No. 60/594,499 to Michael E. Huff filed on 13 Apr. 2005, which is incorporated herein by reference.

The present invention relates to apparatuses and methods of identifying and managing mood, specifically to apparatuses and methods of a user identifying his current mood, the triggers for that mood, and managing that mood.

In U.S. Pat. No. 3,777,413, Zaccheo discloses a method for the systematic investigation of mental phenomena and skills associated with consciousness, behavior, emotions, traits and reactions to stimuli is disclosed which includes the steps of conveying one or more word messages by oral or visual means, associating the message with an identifying indicia and comparing the matches and mismatches of indicia as an indication of the sensitivity or lack thereof to the mental stimuli. The apparatus used to conduct the testing, which can be a serious psychiatric analysis and psychological investigation or a humorous and fascinating parlor game participated in by two or more individuals, comprises, in one embodiment, a set of cards having a plurality of sets of word phrase stimuli thereon with the stimuli of any one set being divided into three or more classes of conservative, moderate and aggressive connotations or meanings. The sets of cards are divided into several categories of human expression relating the senses, time, acts, words, mood, etc. The stimuli on each set of cards are coded in accordance with their class or contrary to the class of other sets so that no preceptive pattern can be established from use in repeated tests or plays and auxiliary means are provided to record the results, score the players and arrive at a final measure of the personal sensitivity, compatibility, analytic skills, psychological reactions, superficial traits and outward manifestations of behavior of one or more participants.

U.S. Pat. No. 6,494,718 discloses a method and product for increasing the awareness of one's self and for assessment and increasing decisiveness in conflict resolution in order to assist individuals who are conflicted by an intimate relationship. The method utilizes a plurality of individual statements which are each associated with one of four (4) different cells. The cells are positioned from lower to higher self-awareness and decisiveness with conflict resolution being enhanced with statements falling within the cells of higher self-awareness and decisiveness. Upon reviewing the individual statements and positioning such statements in appropriate locations on a template, the user becomes aware of his degree of decidedness and the quality of his decision and his level of awareness.

Finally, in U.S. Pat. No. 4,931,934 disclosed is a system and method for measuring the emotional response of a subject to a chosen focus in order to reliably produce objective data representing that emotional response. The subject is put in contact with the chosen focus, and the emotional response of that subject to the chosen focus is determined in order to provide initial response emotions and the intensities of these emotions. The intensity of these same emotions without attention directed to the chosen focus is also evaluated in order to provide tabulated baseline intensities. Utilizing the initial response emotion intensities and the baseline emotion intensities, clarified emotion intensities are determined for use in a variety of decision-making applications.

What is needed is an apparatus and method of identifying and managing mood that solves one or more of the problems described herein and/or one or more problems that may come to the attention of one skilled in the art upon becoming familiar with this specification.

### SUMMARY OF THE INVENTION

The present invention has been developed in response to the present state of the art, and in particular, in response to the problems and needs in the art that have not yet been fully solved by currently available methods and apparatuses for identifying and managing mood. Accordingly, the present invention has been developed to provide an apparatus for identifying and managing mood, including: from seven to ten selectable mood buttons, each representing a mood; selectable trigger buttons, each representing a trigger for the mood; mood suggestions displayed after selection of a mood button and a trigger button; a speaker; and a mode selection button.

The apparatus of may include a wristband. The apparatus may include an on/off switch. The apparatus may include an AC adapter jack. The mood suggestions may be displayed audibly through the speaker.

In another embodiment, the invention includes an apparatus for identifying and managing mood, including only: from seven to ten selectable mood buttons, each representing a mood; selectable trigger buttons, each representing a trigger for the mood; mood suggestions displayed after selection of a mood button and a trigger button; a speaker; and a mode selection button.

The mode selection button may include an on/off switch. The mood suggestions may be displayed audibly through the speaker.

In yet another embodiment, the invention includes a method of identifying and managing mood, comprising the steps of: displaying a selectable mood button, representing a mood; displaying a selectable trigger button, representing a trigger for the mood; and displaying mood suggestions.

The method of identifying and managing mood may include at least one more selectable mood button, representing a different mood. The step of displaying mood suggestions may include displaying mood suggestions audibly through a speaker.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### BRIEF DESCRIPTION OF THE DRAWINGS

In order for the advantages of the invention to be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 illustrates a wrist-mounted apparatus for identifying and managing mood according to one embodiment of the present invention;

FIG. 2 illustrates a desktop apparatus for identifying and managing mood according to one embodiment of the present invention;

FIG. 3 illustrates a method of identifying and managing mood according to one embodiment of the present invention;

FIG. 4 illustrates a perspective view of an apparatus for identifying and managing mood according to one embodiment of the present invention;

FIG. 5 illustrates a top plan view of an apparatus for identifying and managing mood according to one embodiment of the present invention;

FIG. 6 illustrates a front plan view of an apparatus for identifying and managing mood according to one embodiment of the present invention;

FIG. 7 illustrates a top plan view of a wrist-mounted apparatus for identifying and managing mood according to one embodiment of the present invention;

FIG. 8 illustrates side plan view of a wrist-mounted apparatus for identifying and managing mood according to one embodiment of the present invention;

FIG. 9 illustrates a top plan view of a wrist-mounted apparatus for identifying and managing mood according to one embodiment of the present invention; and

FIG. 10 illustrates side plan view of a wrist-mounted apparatus for identifying and managing mood according to one embodiment of the present invention.

### DETAILED DESCRIPTION OF THE INVENTION

For the purposes of promoting an understanding of the principles of the invention, reference will now be made to the exemplary embodiments illustrated in the drawings, and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

Reference throughout this specification to “one embodiment,” “an embodiment,” or similar language means that a particular feature, structure, or characteristic described in connection with the embodiment is included in at least one embodiment of the present invention. Thus, appearances of the phrases “one embodiment,” “an embodiment,” and similar language throughout this specification may, but do not necessarily, all refer to the same embodiment, different embodiments, or component parts of the same or different illustrated invention. Additionally, reference to the wording “an embodiment,” or the like, for two or more features, elements, etc. does not mean that the features are related, dissimilar, the same, etc. The use of the term “an embodiment,” or similar wording, is merely a convenient phrase to indicate optional features, which may or may not be part of the invention as claimed.

Each statement of an embodiment is to be considered independent of any other statement of an embodiment despite any use of similar or identical language characterizing each embodiment. Therefore, where one embodiment is identified as “another embodiment,” the identified embodiment is independent of any other embodiments characterized by the language “another embodiment.” The independent embodiments are considered to be able to be combined in whole or in part one with another as the claims and/or art may direct, either directly or indirectly, implicitly or explicitly.

Finally, the fact that the wording “an embodiment,” or the like, does not appear at the beginning of every sentence in the specification, such as is the practice of some practitioners, is merely a convenience for the reader's clarity. However, it is the intention of this application to incorporate by reference the phrasing “an embodiment,” and the like, at the beginning of every sentence herein where logically possible and appropriate.

Many of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of executable code may, for instance, comprise one or more physical or logical blocks of computer instructions which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, a module of executable code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network.

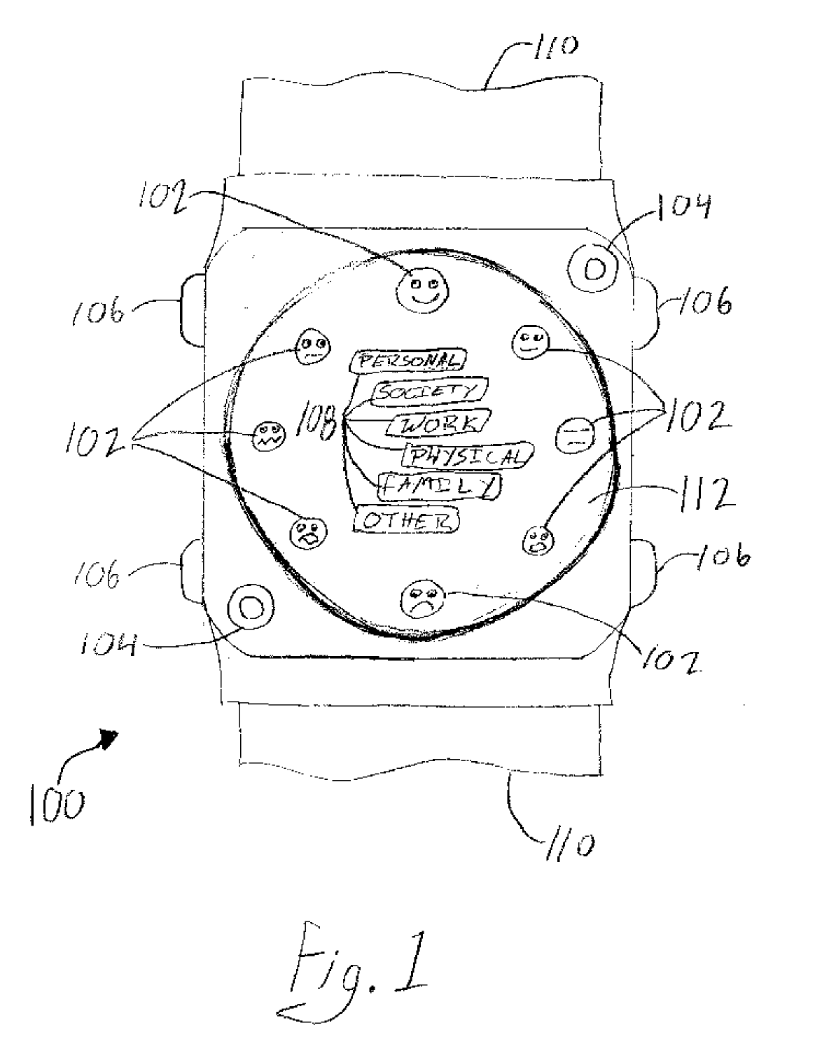

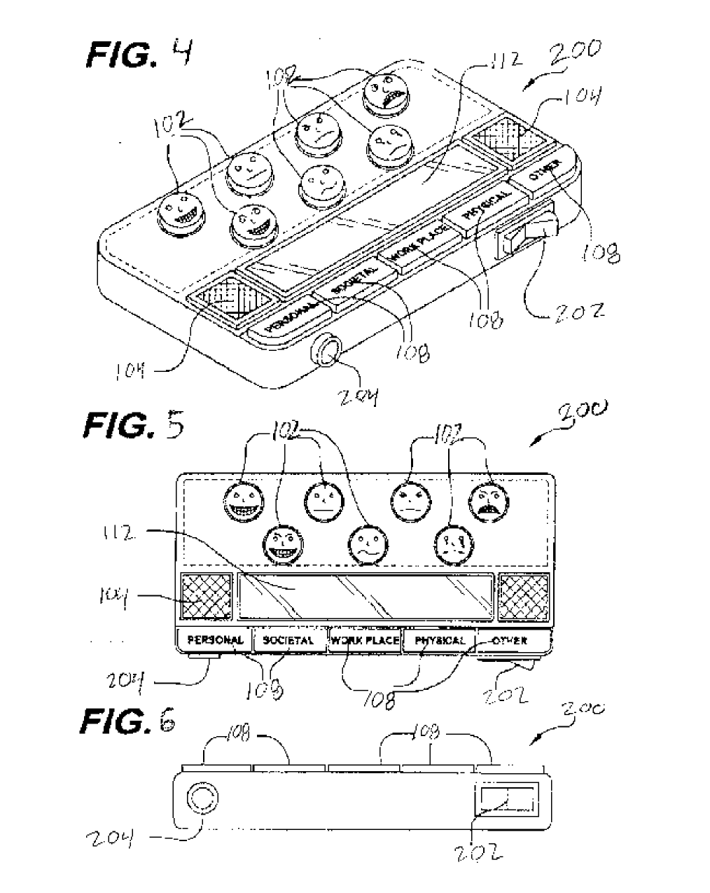

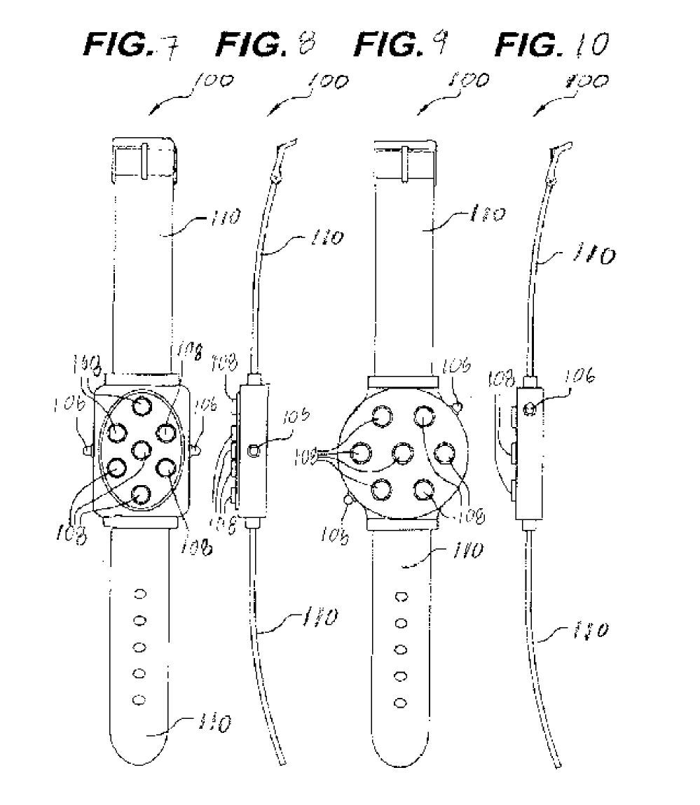

FIGS. 1 and 7-10 illustrate a wrist-mounted apparatus 100 for identifying and managing mood according to several embodiments of the present invention. The apparatus includes a wrist-mounted device 100 that is capable of being kept or mounted on the wrist, much like a wristwatch. FIGS. 2 and 4-6 illustrate a desk-mounted or pocket apparatus 200 according to several embodiments of the present invention. The apparatus 100, 200 includes at least one selectable mood button 102, each representing a mood. In one embodiment, the apparatus 100, 200 may include from about 7 to about 10 selectable mood buttons 102. The moods represented by the selectable mood buttons 102 may include any that may be experienced. Some non-limiting examples of these moods includes, happy, sad, ecstatic, gloomy, depressed, excited, gleeful, content, satisfied, angry, and so forth. The selectable mood buttons 102 may be indicated by words, pictures, and so forth. The words, pictures, and so forth may represent the mood identified with the selectable mood buttons 102.

The apparatus 100, 200 also may include selectable mood trigger buttons 108. In one embodiment, the mood trigger buttons 108 correspond to certain triggers that trigger certain moods. For example, one mood trigger button 108 may include any person, place, thing, or event that could trigger a mood. Some non-limiting examples of mood triggers include personal, societal, work, physical, family, other, and so forth. The selectable mood trigger buttons 108 may be selected to indicate what trigger is the cause of the selected mood.

The selectable mood trigger buttons 108 and the mood buttons 102 may be any buttons known in the art. In one embodiment, the buttons 102, 108 include physical buttons such as those on a wristwatch, calculator, or other electronic device. In another embodiment, the buttons 102, 108 may include icons that are selectable upon touching, pressing, or contact with a stylus, such as the icons found on a personal data assistant or personal computer.

The apparatus 100, 200 also may include at least one speaker 104. In one embodiment, the apparatus 100, 200 includes two speakers 104. The speakers may be located anywhere on the apparatus 100, 200. In one embodiment, the speaker 104 is mounted on the face of the apparatus 100, 200. The speaker may be used to display suggestions generated by the apparatus 100, 200, as described below.

The apparatus 100, 200 may include a screen 112. The screen 112 may be any capable of displaying characters. The screen 112 may include, for example, a liquid crystal display (LCD), plasma screen, television tube screen, and so forth. In one preferred embodiment, the screen 112 includes a LCD screen. The screen may react to touching or depressing. In one example, the screen uses the same technology as the screen of a personal data assistant, wherein a finger or stylus may be used to select certain areas of the screen.

The apparatus 100, 200 may also include mode buttons 106. The mode buttons 106 may be used to change modes of the apparatus. For example, the apparatus 100, 200 may include alternative modes such as a watch mode, wherein the time is displayed, a calendar mode, wherein a calendar is displayed, and/or other modes known in the art. In one embodiment, the mode buttons 106 of the apparatus 200 includes an on/off switch 202 that turns the apparatus 200 on or off. In another embodiment, one of the mode buttons 106 of the apparatus 100 includes an on/off switch that turns the apparatus 100 on or off.

The apparatus 200 may also include an AC jack 204. The AC jack 204 may be configured such that the apparatus 200 may receive electrical power from a power source other than internal batteries.

In one embodiment, the apparatus 100, 200, displays mood suggestions once a mood and a trigger have been selected. The mood suggestions may be displayed on the screen 112, or displayed audibly through the speaker 104, or both. The apparatus may perform some calculations such that the mood suggestions correspond to the selected mood and trigger. The mood suggestions may be pre-recorded and stored in the apparatus 100, 200. In one embodiment, the apparatus 100, 200 includes a module wherein the selection of the mood the trigger by the user results in an automatic selection of a mood suggestion. In another embodiment a module for the selection of the mood suggestion includes selection of a group of mood suggestions, and the random display of one of the mood suggestions in the group of mood suggestions. In this embodiment, the user would likely not receive the same mood suggestion each time the same mood and triggers are selected.

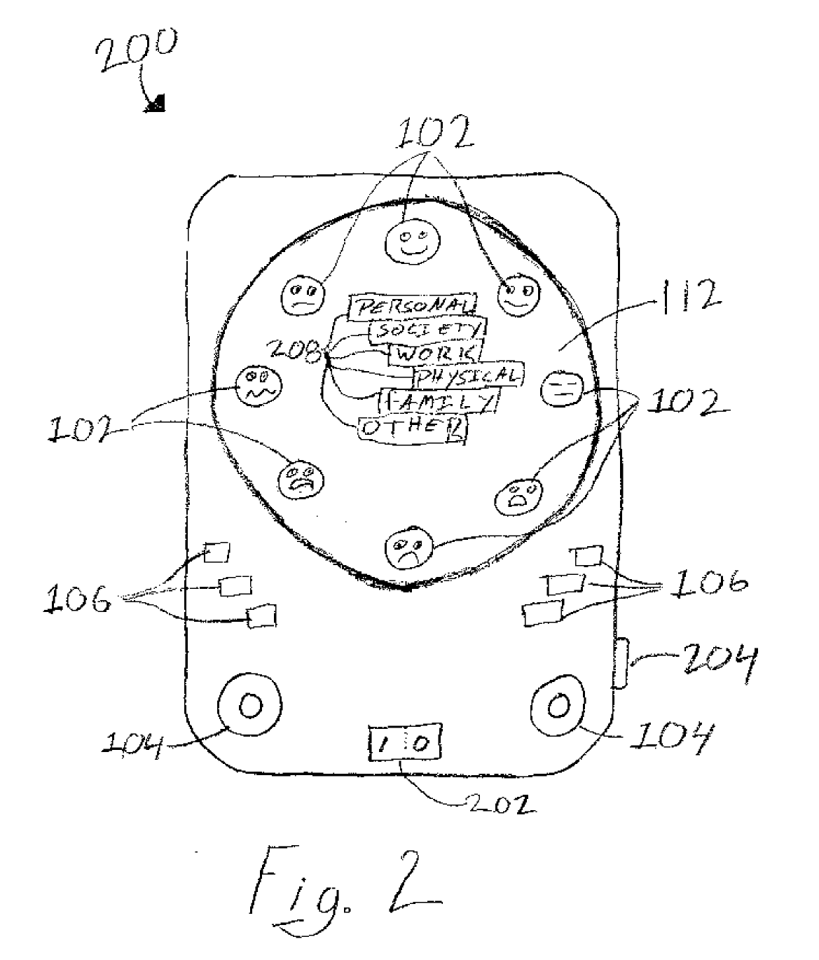

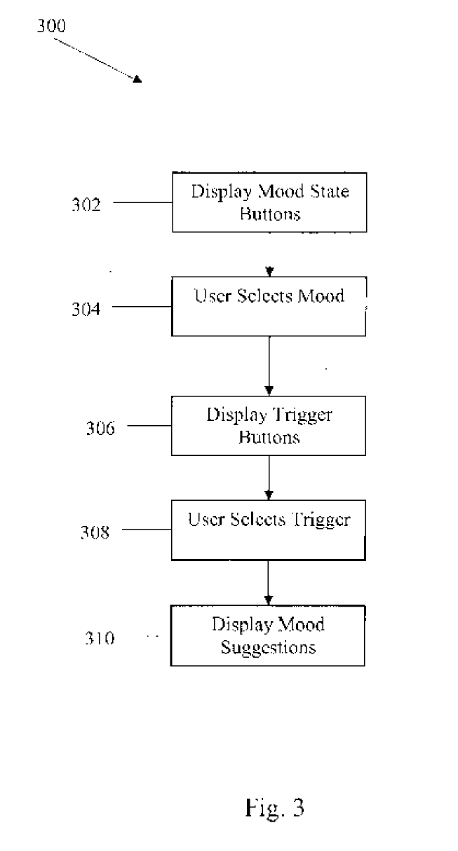

FIG. 3 illustrates a flow diagram of a method of identifying and modifying mood 300 according to one embodiment of the present invention. The method 300 includes the step of displaying mood state buttons 302. The display may be on an apparatus 100, 200 (see FIGS. 1, 2, and 4-10) as described above. The display may be by any method as described above. The mood buttons may correspond to moods, as described above.

The method 300 includes the step of the user selecting mood 304. In this step, the user may select the mood state button that corresponds to the mood of the user. The selection may be made by any method as described above.

The method 300 includes the step of displaying trigger buttons 306. The trigger buttons may be displayed on an apparatus 100, 200 (see FIGS. 1, 2, and 4-10) as described above. The display may be by any method as described above. The trigger buttons may correspond to triggers to moods, as described above.

The method 300 includes the step of the user selecting the trigger 308. In this step, the user may select the trigger button that corresponds to the trigger to the mood of the user. This selection may be made by any method as described above.

The method 300 also includes the step of displaying mood modification suggestions 310. The mood suggestions may be used to alter or preserve the mood of the user. The mood suggestions may be displayed on a display screen, or audibly through speakers, or both. Some non-limiting examples of mood suggestions includes “keep at it,” “keep your head up,” “go get 'em!” “just put one foot in front of the other,” and so forth.

The method and apparatus herein described may be used to manage the mood of a user. The management of mood may be by prompting an individual to self-reflect, identify mood-state, identify emotional triggers, read or hear mood modification suggestions, and implement mood modification suggestions. The method and apparatus may be used in child and patient care settings, human resources, armed forces, management and employee development programs, educational settings, professional athletics, religious settings, law enforcement, anger management programs, family and marital art therapy programs, psychological therapy, to aid the hard of hearing and visually impaired in communicating, to increase self-awareness, to aid in actors technique, and/or as a quiz game.

It is understood that the above-described preferred embodiments are only illustrative of the application of the principles of the present invention. The present invention may be embodied in other specific forms without departing from its spirit or essential characteristics. The described embodiment is to be considered in all respects only as illustrative and not restrictive. The scope of the invention is, therefore, indicated by the appended claim rather than by the foregoing description. All changes which come within the meaning and range of equivalency of the claims are to be embraced within their scope.

It is also envisioned that the apparatus may be worn on a wrist, kept on a desk, wall-mounted, door-mounted, and so forth. The apparatus and device may have a musical playback. It could be constructed with GPS tacking. The apparatus may include an earpiece or headset. There may be a language adaptation module to translate any of the functions into predetermined languages. There may be a password security module. There could be a voice activation feature. There may be wireless internet access. There could be a module to log mood activities. There could be a module with a scheduler and reminders

It is expected that there could be numerous variations of the design of this invention. An example is that the apparatus may include other user inputs. The inputs may include a biometric sensor. The sensor may input, for example, a user temperature, skin conductance, heart rate, hormone levels, neurological activities, and so forth. The method and apparatus may include these inputs in the calculations of choosing the mood suggestion to display.

Thus, while the present invention has been fully described above with particularity and detail in connection with what is presently deemed to be the most practical and preferred embodiment of the invention, it will be apparent to those of ordinary skill in the art that numerous modifications, including, but not limited to, variations in size, materials, shape, form, function and manner of operation, assembly and use may be made, without departing from the principles and concepts of the invention as set forth in the claims.

What is claimed is:

1. An apparatus for identifying and managing mood, comprising:
from seven to ten selectable mood buttons, each representing a mood;
selectable trigger buttons, each representing a trigger for the mood;
mood suggestions displayed after selection of a mood button and a trigger button;
a speaker; and
a mode selection button.

2. The apparatus of claim 1, further comprising a wristband.

3. The apparatus of claim 1, further comprising an on/off switch.

4. The apparatus of claim 1, further comprising an AC adapter jack.

5. The apparatus of claim 1, wherein the mood suggestions comprise displayed audibly through the speaker.

6. An apparatus for identifying and managing mood, consisting of:
from seven to ten selectable mood buttons, each representing a mood;
selectable trigger buttons, each representing a trigger for the mood;
mood suggestions displayed after selection of a mood button and a trigger button;
a speaker; and
a mode selection button.

7. The apparatus of claim 1, wherein the mode selection button includes an on/off switch.

8. The apparatus of claim 1, wherein the mood suggestions comprise displayed audibly through the speaker.

9. A method of identifying and managing mood, comprising the steps of:
displaying a selectable mood button, representing a mood;
displaying a selectable trigger button, representing a trigger for the mood; and
displaying mood suggestions.

10. The method of identifying and managing mood of claim 9, further comprising at least one more selectable mood button, representing a different mood.

11. The method of identifying and managing mood of claim 9, wherein the step of displaying mood suggestions includes displaying mood suggestions audibly through a speaker.
